(19) United States
(12) Patent Application Publication
(10) Pub. No.: US 2024/0081719 A1
(43) Pub. Date: Mar. 14, 2024

(54) SYSTEMS AND METHODS FOR ATTENTION DEFICIT DETECTION FRAMEWORK

(71) Applicant: BITSPHI DIAGNOSIS SL, Madrid (ES)

(72) Inventors: Álvaro Medrano López, Madrid (ES); Miguel Blanco Carmona, Madrid (ES); Fernando Maestú Unturbe, Madrid (ES)

(73) Assignee: BITSPHI DIAGNOSIS SL, Madrid (ES)

(21) Appl. No.: 17/931,086

(22) Filed: Sep. 9, 2022

(51) Int. Cl. A61B 5/369; A61B 5/00; G16H 50/20

(52) U.S. Cl. CPC A61B 5/369; A61B 5/0042; A61B 5/4064; A61B 5/72; G16H 50/20

(57) ABSTRACT

Described herein are methods, systems, and computer-readable storage media for automated detection of attention deficit hyperactivity disorder (ADHD). Techniques include providing stimuli to a user to activate a plurality of brain regions representing an attention network by displaying information with a first condition that causes a reaction and a second condition as an exception to the first condition. Techniques further include retrieving a plurality of signals from the plurality of brain regions for each of the stimuli, wherein each signal of the plurality of signals is accessed over a period of time that starts when the information is displayed and ends when the reactions of the user are captured. Further, the attention deficit detection system evaluates the signals to detect ADHD based on the level of connectivity within the attention network when each signal of the plurality of signals corresponds to the one or more stimuli.

### TECHNICAL FIELD

This disclosure relates to an attention deficit detection framework. In some embodiments, for example, this disclosure relates to systems and methods for defining tasks of content provided as stimuli used to obtain a reaction from a user, and evaluating the generated brain signals for attention deficiencies.

### BACKGROUND

Attention deficit hyperactivity disorder (ADHD) is a neurodevelopmental disorder characterized by inappropriate levels of inattention and hyperactivity or impulsivity, whose onset usually occurs during childhood with a relative high prevalence. ADHD is associated with neurocognitive deficits and impaired psychosocial-vocational functioning that can be highly debilitating and have adverse consequences for both patients and their environments, leading, in the worst cases, to increased mortality and suicide (Barkley 2002; Impey and Heun 2012; Erskine et al. 2016; Nourredine et al. 2021; Barkley and Dawson 2022; Catala-Lopez et al. 2022). It is therefore of crucial importance to correctly diagnose ADHD, both to establish action plans of intervention as well as to guarantee the validity of clinical research. Currently, the gold standard for diagnosing ADHD consists of a clinical interview, behavioral observations, and neuropsychological testing and analysis of different drug effects (Gualtieri & Johnson 2005). This methodology relies on observable behavioral patterns, disregarding the neural mechanisms that underlie such responses, and is inherently conditioned by the examiners' and parents' perception as well as their knowledge and beliefs about the deficit. Furthermore, the symptoms of ADHD are a dynamic process in which neurobiological deficits that present a relatively slow evolution are modulated by maturational and compensatory processes, which poses a challenge to its diagnosis due to the great heterogeneity of the deficit, both cross-sectional between subjects and longitudinal intra-subjects. Hence, the current approach suffers from arbitrariness, lack of reproducibility, and dissociation from pathophysiology, which highlights the need to improve the nosology of ADHD by searching for the connections between its symptomatology and neurobiology.

Thus, there is a need to find solutions that avoid arbitrariness and provide with high probability consistent results for indication of attention deficiencies such as ADHD. Such solutions would advantageously help reproduce the same results over time and avoid incorrect assumptions. Further technical solutions are described in the example embodiments below to aid in producing consistent predictable results.

### SUMMARY

Certain embodiments of the present disclosure relate to a non-transitory computer-readable medium including instructions that, when executed by at least one processor, cause the at least one processor to perform operations for automated detection of ADHD. The operations may include providing one or more stimuli to a user to activate a plurality of brain regions representing an attention network, wherein the one or more stimuli include displaying information with a first condition that causes a reaction and a second condition as an exception to the first condition; retrieving a plurality of signals from the plurality of brain regions for each of the one or more stimuli, wherein each signal of the plurality of signals is accessed over a period of time, wherein the period of time starts when the information is displayed and ends when the reactions of the user are captured; and evaluating the plurality of signals to detect ADHD based on the level of connectivity within the attention network when the signal corresponds to the one or more stimuli.

According to some disclosed embodiments, displaying information with a first condition that causes a reaction and a second condition as an exception to the first condition may further comprise presenting an instance of data matching a first task, wherein the first task includes the first condition for an expectation of an active state and a silent state of a first reaction, the second condition restricting the expectation of the active state of the first reaction.

According to some disclosed embodiments, the first reaction may further comprise displaying the information on a screen meeting the first condition for an expectation of the active state and the silent state of the first reaction, and waiting for a threshold period for any reaction shared by the user.

According to some disclosed embodiments, the first reaction is at least one of clicking a pointing device, pressing a button, or taking no action for a time threshold.

According to some disclosed embodiments, evaluating the plurality of signals to detect ADHD based on the level of connectivity within the attention network may further comprise filtering alpha band oscillation signals of two or more regions of the plurality of brain regions of the user upon receiving the first reaction and determining connectivity between two regions of the brain using the alpha band oscillation signals, and comparing the level of connectivity between the two regions of the brain of the user to the level of connectivity of a healthy control group of users when the first condition of the first task satisfies the active state, and the second condition is negative, wherein if the level of connectivity of the user is less than the level of connectivity of the healthy control group of users, then the user is indicative of having ADHD.

According to some disclosed embodiments, evaluating the plurality of signals to detect ADHD based on the level of connectivity within the attention network may further comprise filtering alpha band oscillation signals of two or more regions of the plurality of brain regions of the user upon receiving the first reaction and determining connectivity between two regions of the brain using the alpha band oscillation signals, and comparing the level of connectivity between the two regions of the brain of the user to the level of connectivity of a healthy control group of users when the first condition of the first task satisfies the silent state, or the second condition is positive, wherein if the level of connectivity of the user is less than the level of connectivity of the healthy control group of users, then the user is indicative of having ADHD.

According to some disclosed embodiments, the operations may further comprise filtering beta band oscillation signals of two or more brain regions upon receiving the first reaction and determining connectivity between two regions of the brain using beta band oscillation signals, and comparing the level of connectivity between the two regions of the brain of the user to the level of connectivity of a healthy control group of users when the first condition of the first task satisfies the silent state, or the second condition is positive, wherein if the level of connectivity of the user is greater than the level of connectivity of the beta band oscillation signals of the healthy control group of users, then the user is indicative of having ADHD.

According to some disclosed embodiments, the operations may further comprise filtering alpha band oscillation signals of two or more brain regions upon receiving the first reaction and determining connectivity between two regions of the brain using the alpha band oscillation signals, determining the level of connectivity between the two regions of the brain of the user when the first condition of the first task satisfies the silent state and when the first condition of the first task satisfies the active state, and comparing the reduction in the level of connectivity of the user between when the first task satisfies the silent state and when the first task satisfies the active state to the reduction in the level of connectivity of a healthy control group of users between when the first task satisfies the silent state and when the first task satisfies the active state, wherein if the reduction in the level of connectivity of the user is less than the reduction in the level of connectivity of the healthy control group, then the user is indicative of having ADHD.

According to some disclosed embodiments, evaluating the plurality of signals to detect ADHD based on the level of connectivity in the attention network may further comprise combining signal data from the plurality of signals, wherein combining signal data includes determining the increase in the level of connectivity compared to an existing level of connectivity of a region of the plurality of regions or the reduction in the level of connectivity from the existing level of connectivity of the region of the plurality of regions.

According to some disclosed embodiments, displaying information with a first condition includes displaying a category of text or graphic.

According to some disclosed embodiments, the second condition includes displaying the category of text or graphic in a particular color.

According to some disclosed embodiments, the one or more stimuli may further comprise displaying second information with the first condition that causes a reaction, the second condition as an exception to the first condition, and a third condition as an exception to the second condition.

According to some disclosed embodiments, displaying second information with the first condition that causes a reaction, the second condition as an exception to the first condition, and a third condition as exception to the second condition may further comprise presenting an instance of data matching a second task, wherein the second task includes the first condition for an expectation of an active state and a silent state of a second reaction, the second condition restricting expectation of the active state of the second reaction, the third condition that is an exception to the second condition for an expectation of the silent state of the second reaction.

According to some disclosed embodiments, the second reaction may further comprise displaying information on a screen meeting the first condition for an expectation of the active state and the silent state of the second reaction, and waiting for a threshold period for any input shared by the user.

According to some disclosed embodiments, the third condition may include selecting a sub-category of a category of text or graphic meeting the first condition and displaying the sub-category of the text or graphic.

According to some disclosed embodiments, the second reaction is at least one of clicking a pointing device, pressing a button, or taking no action for a threshold time.

According to some disclosed embodiments, the operations may further comprise filtering beta band oscillation signals of two or more regions of the plurality of brain regions of the user upon receiving the second reaction and determining connectivity between two regions of the brain using the beta band oscillation signals; and comparing the level of connectivity between the two regions of the brain of the user to the level of connectivity of a healthy control group of users when the first condition of the second task satisfies the active state, the second condition is negative and the third condition is negative, or when the first condition of the second task satisfies the active state, the second condition is positive and the third condition is positive, wherein if the level of connectivity of the user is greater than the level of connectivity of the healthy control group of users, then the user is indicative of having ADHD.

According to some disclosed embodiments, the operations may further comprise filtering theta band oscillation signals of two or more regions of the plurality of brain regions of the user upon receiving the second reaction from the user and determining connectivity between two regions of the brain using the theta band oscillation signals, comparing the level of connectivity between the two regions of the brain of the user to the level of connectivity of a healthy control group of users when the first condition of the second task satisfies the active state, the second condition is negative and the third condition is negative, or when the first condition of the second task satisfies the active state, the second condition is positive and the third condition is positive, wherein if the signal data of the level of connectivity of the user is less than the level of connectivity of the healthy control group of users, then the user is indicative of having ADHD.

According to some disclosed embodiments, the operations may further comprise filtering alpha band oscillation signals of two or more regions of the plurality of brain regions of the user upon receiving the second reaction from the user and determining connectivity between two regions of the brain using the oscillation signals. The operations may further comprise determining a variation in the level of connectivity between the two regions of the brain of the user when presenting the instance of data for the second task. For such determination of variation, comparing the level of connectivity when the first condition of the second task satisfies the active state, the second condition of the second task is positive and the third condition of the second task is positive and the level of connectivity when the first condition of the first task satisfies the active state and the second condition of the first task is positive. The operation further comprises comparing the variation in the level of connectivity of the user to the variation in the level of connectivity of a healthy control group of users, wherein if the variation in the level of connectivity of the user is different than the variation in the level of connectivity of the healthy control group of users, then the user is indicative of having ADHD.

According to some disclosed embodiments, the operations may further comprise filtering alpha, beta, and theta band oscillation signals of two or more regions of the plurality of brain regions of the user upon receiving the first reaction and the second reaction and determining connectivity between regions of the brain using the alpha, beta, and theta band oscillation signals; and evaluating the probability of the detection of ADHD based on the comparison of the level of connectivity, reduction in the level of connectivity, and the variation in the level of connectivity between the regions of the brain of the user to the level of connectivity of a healthy control group when varying the first condition and the second condition of the first task, and the first condition, the second condition, and the third condition of the second task.

Certain embodiments of the present disclosure relate to a method performed by a system for automated detection of ADHD. The method can include providing one or more stimuli to a user to activate a plurality of brain regions representing an attention network, wherein the one or more stimuli include displaying information with a first condition that causes a reaction and a second condition as an exception to the first condition; retrieving a plurality of signals from the plurality of brain regions for each of the one or more stimuli, wherein each signal of the plurality of signals is accessed over a period of time, wherein the period of time starts when the information is displayed and ends when the reactions of the user are captured; and evaluating the plurality of signals to detect ADHD based on the level of connectivity in the attention network when the signal corresponds to the one or more stimuli.

Certain embodiments of the present disclosure relate to an attention deficit detection system (ADDS). The attention deficit detection system includes one or more processors executing processor-executable instructions stored in one or more memory devices to perform a method. The method may include providing one or more stimuli to activate a plurality of brain regions representing an attention network, wherein the one or more stimuli include displaying information with a first condition that causes a reaction and a second condition as an exception to the first condition; retrieving a plurality of signals from the plurality of brain regions for each of the one or more stimuli, wherein each signal of the plurality of signals is accessed over a period of time, wherein the period of time starts when the information is displayed and ends when the reactions of the user are captured; and evaluating the plurality of signals to detect ADHD based on the level of connectivity in the attention network when the signal corresponds to the one or more stimuli.

Certain embodiments of the present disclosure relate to an attention deficit detection system. The attention deficit detection system includes one or more processors executing processor-executable instructions stored in one or more memory devices to perform a method. The method may include receiving first signal data from a user; determining a first plurality of time series based on the first signal data, wherein each of the first plurality of time series corresponds to a respective source position located inside a cranial cavity of the user; calculating a first correlation value for a first pair of time series, the first pair of time series being included in the determined first plurality of time series; generating a score based on the first correlation value, the score being indicative of the patient having a cognitive impairment, such as attention deficiencies or ADHD; and outputting the generated score.

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIG. 1 is a block diagram showing various exemplary components of an attention deficit detection system for the accurate detection of ADHD, according to some embodiments of the present disclosure.

FIGS. 2A-B are tabular representations of exemplary tasks for the detection of ADHD and its probabilities, according to some embodiments of the present disclosure.

FIG. 2C illustrates conditional probabilities of each output based on a cascade model, according to some embodiments of the present disclosure.

FIG. 3 illustrates connection networks between regions of the brain, according to some embodiments of the present disclosure.

FIG. 4 illustrates a schematic diagram of an exemplary server of a distributed system, according to some embodiments of the present disclosure.

FIG. 5 is a flowchart depicting operations of an exemplary method for detection of ADHD, according to some embodiments of the present disclosure.

FIG. 6 is a flowchart depicting operations of an exemplary method for detection of ADHD, according to some embodiments of the present disclosure.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are neither constrained to a particular order or sequence nor constrained to a particular system configuration. Additionally, some of the described embodiments or elements thereof may occur or be performed simultaneously, at the same point in time, or concurrently. Reference will now be made in detail to the disclosed embodiments, examples of which are illustrated in the accompanying drawings. Unless explicitly stated, “sending” and “receiving” as used herein are understood to have broad meanings, including sending or receiving in response to a specific request or without such a specific request. These terms thus cover both active forms and passive forms of sending and receiving.

Systems and methods consistent with the present disclosure are directed to efficient and accurate detection of ADHD in individuals. Systems and methods described below include techniques of presenting simple tasks to accurately detect the presence of ADHD and the probability of certainty of ADHD. The invention further identifies a metric for modeling the tasks and calculating the probability of certainty of ADHD. In some embodiments, the disclosed techniques include multiple tasks for presenting content with different conditions that cause a reaction to the content. As described below, specialized tasks with reaction conditions can result in various technological improvements in the accuracy of ADHD detection using the underlying system, hardware, and software, and other applications being executed on the underlying hardware and software.

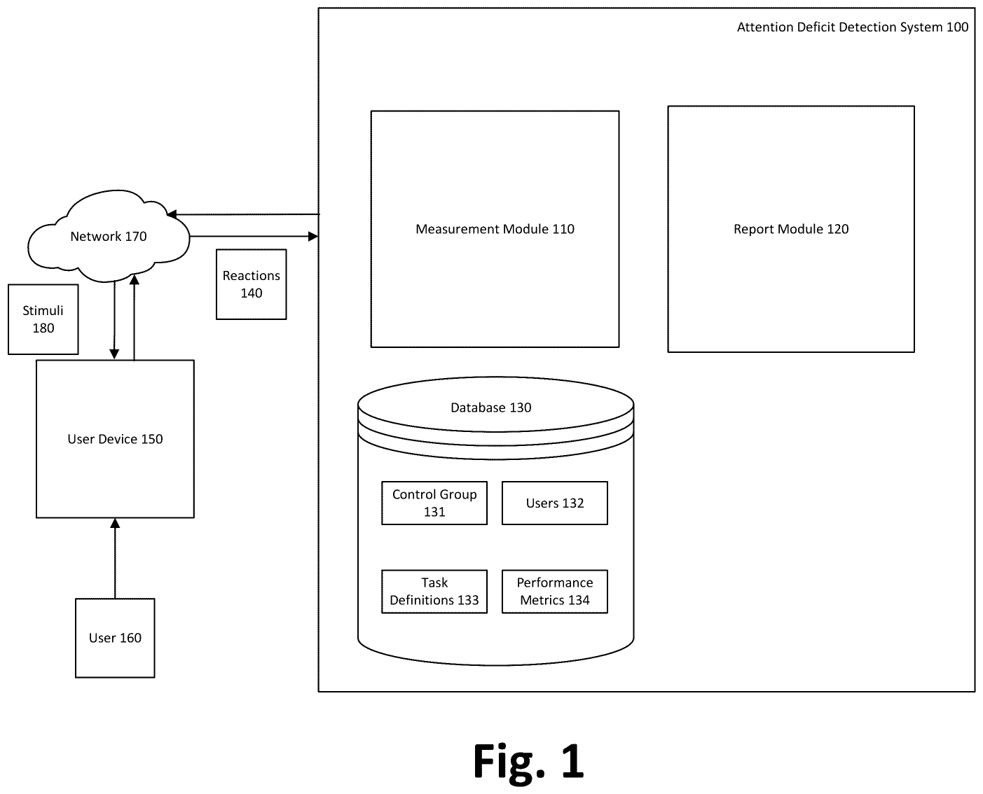

FIG. 1 is a block diagram showing various exemplary components of an attention deficit detection system (ADDS) 100 for the accurate detection of ADHD, according to some embodiments of the present disclosure. Detection of ADHD may include evaluating users of ADDS 100 to detect the presence of ADHD and the percentage of certainty of ADHD. A measure of indication of ADHD may be based on an amount of matches between the user's reactions and the expected results of the tasks. In some embodiments, ADDS 100 may measure the amount of task reaction deviation from the task reactions defined by a user of ADDS 100 as part of the initialization of ADDS 100. A user of ADDS 100 may define attention-level measures of healthy individuals when configuring ADDS 100. For example, user 160 may configure ADDS 100 by supplying a text configuration file to be parsed by ADDS 100.

As illustrated in FIG. 1, ADDS 100 may include measurement module 110 to evaluate the attention levels of individuals and database 130 to store the evaluated attention levels in performance metrics 134. Measurement module 110 may help determine attention levels using data from database 130. Database 130 may aid in evaluating attention levels based on control group 131 associated with users 132 through task definitions 133. Report module 120 may help evaluate the attention levels to determine the attention deficiencies of users 132 associated with control group 131. ADDS 100 may utilize reactions 140 provided using user device 150 to determine the attention levels of control group 131 and users 132. User device 150 may be a processor or a complete computing device, such as a laptop, desktop computer, mobile device, smart home appliance, IoT device, etc.

Measurement module 110 may measure the attention levels of a user (e.g., user 160) by measuring the oscillatory signals in different regions of the brain of the user. Measurement module 110 may measure user 160's attention levels by determining the level of connectivity between different regions of the brain of user 160. Measurement module 110 may measure oscillatory signals upon receiving reactions 140 from user 160 from user device 150. ADDS 100 may be configured to allow measurement module 110 to collect signals from different brain regions based on reactions. The configurations may include whether to collect brain signals and when to collect brain signals, and from which regions to collect brain signals. A user of ADDS 100 may configure measurement module 110 using a configuration provided using user device 150 over network 170.

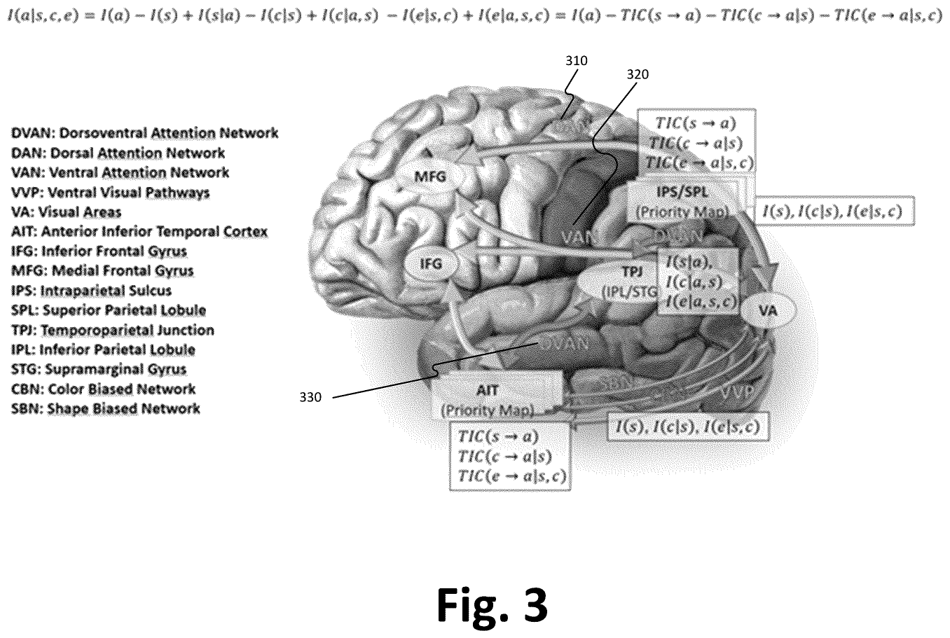

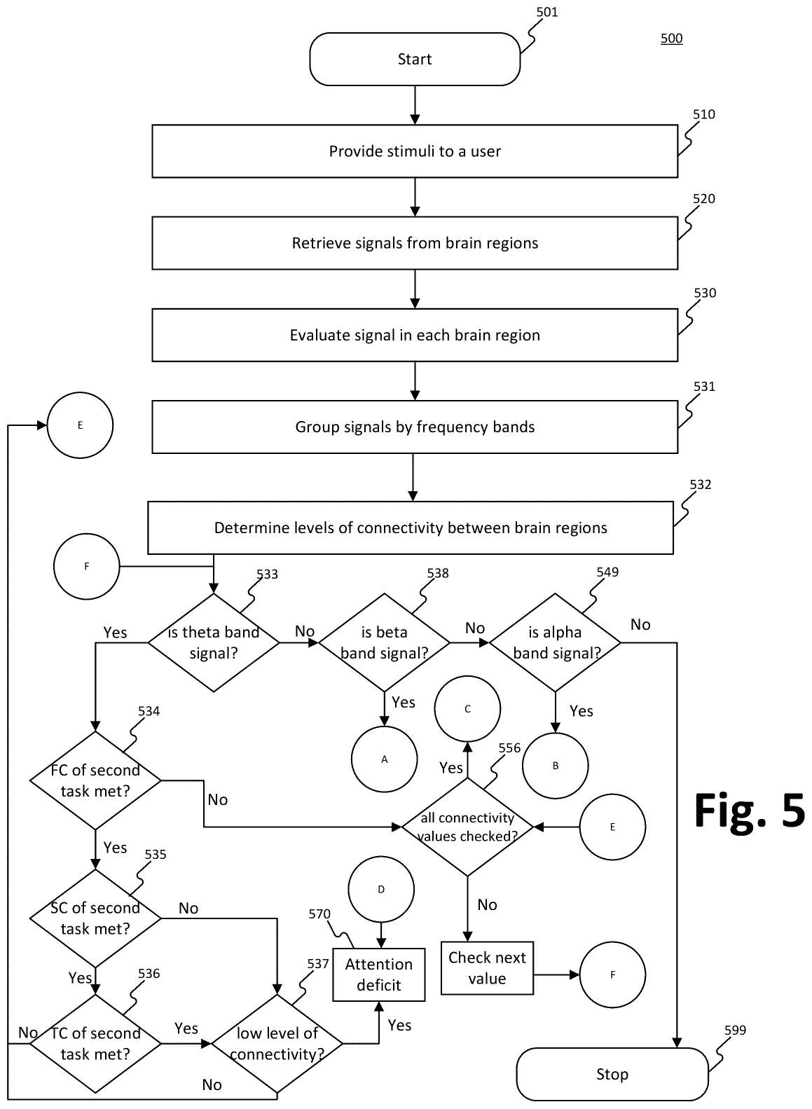

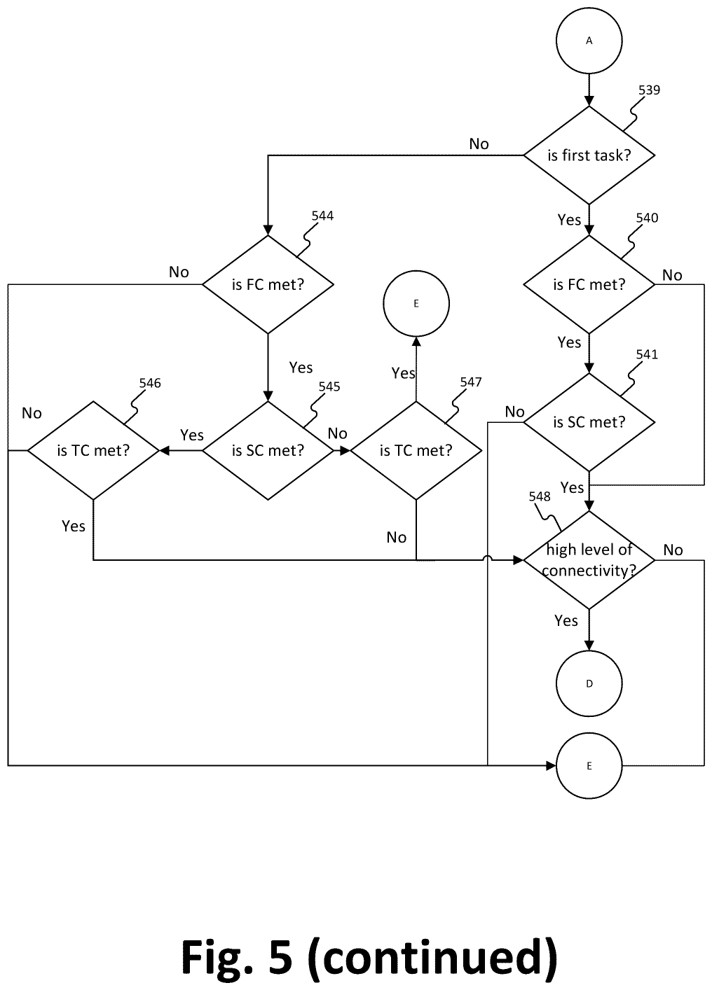

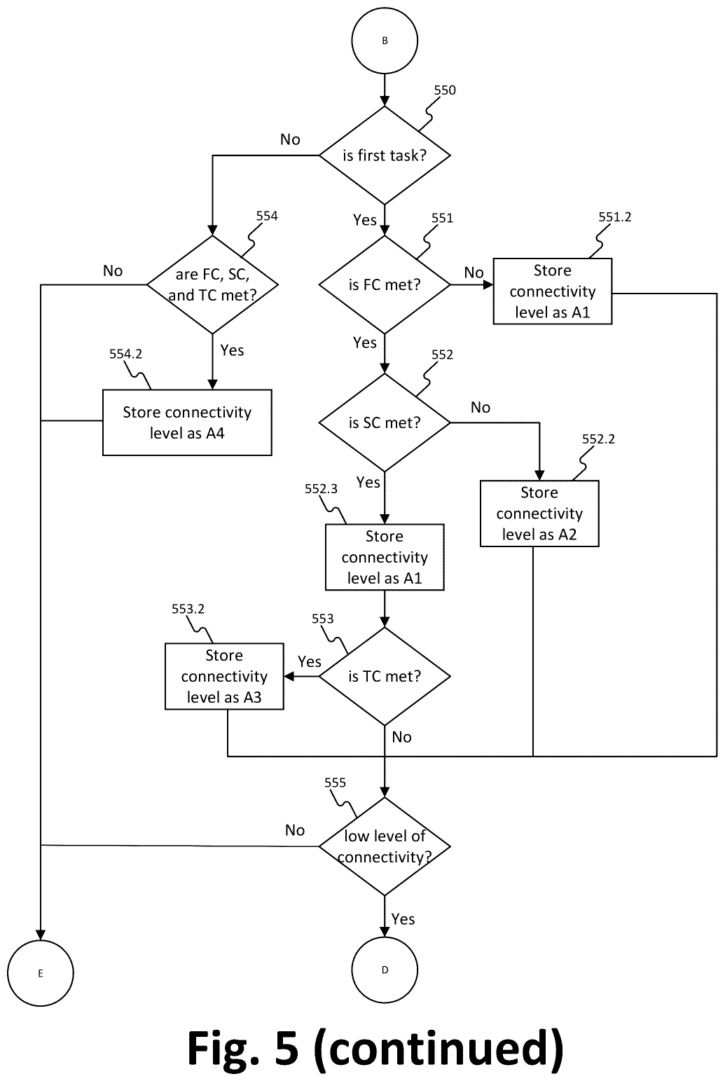

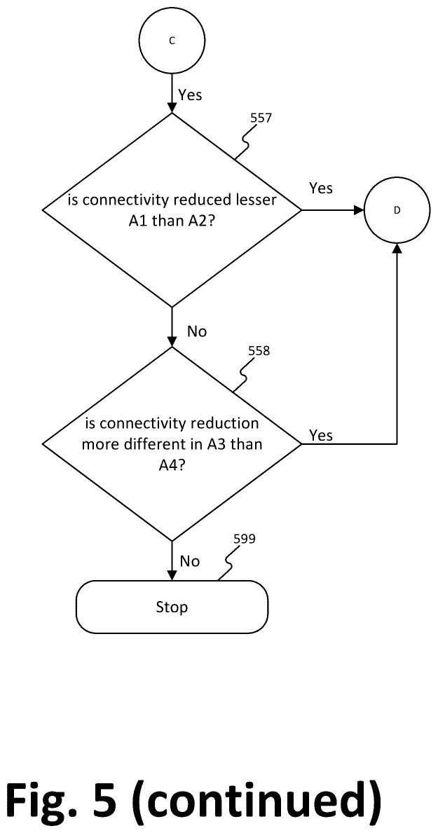

Report module 120 may evaluate the measured signals to determine the presence of ADHD and calculate the percentage of certainty of ADHD. Report module 120 may evaluate signals by measuring signal connectivity. In some embodiments, report module 120 may evaluate signals by comparing determined levels of connectivity. A detailed description of various evaluation techniques is provided in the descriptions of FIG. 3 and FIG. 5 below.

In various embodiments, database 130 may take several different forms. For example, database 130 may be an SQL database or an NoSQL database, such as those developed by MICROSOFT™, REDIS, ORACLE™, CASSANDRA, or MYSQL, or various other types of databases, including data returned by calling a web service, data returned by calling a computational function, sensor data, IoT devices, or various other data sources. Database 130 may store data that is used or generated during the operation of applications, such as data generated by measurement module 110. For example, if measurement module 110 is configured to evaluate reactions 140, then it may access task definitions 133 to share stimuli 180 and measure signals using levels of connectivity upon receiving reactions 140 and store them as performance metrics 134. Similarly, if report module 120 is configured to determine ADHD, then report module 120 may access performance metrics 134 to retrieve the evaluated signal levels of connectivity associated with a user (e.g., user 160) for stimuli against the levels of connectivity of signals for the same stimuli provided to a control group 131. In some embodiments, database 130 may be fed data from an external source (e.g., server, database, sensors, IoT devices, etc.). In some embodiments, database 130 may provide data storage for a distributed data processing system (e.g., Hadoop Distributed File System, Google File System, ClusterFS, and/or OneFS).

Network 170 may take various forms. For example, network 170 may include or utilize the Internet, a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, or other types of network communications. In some embodiments, network 170 may include an on-premises (e.g., LAN) network, while in other embodiments, network 170 may include a virtualized (e.g., AWS™, Azure™, IBM Cloud™ etc.) network. Further, network 170 may in some embodiments be a hybrid on-premises and virtualized network, including components of both types of network architecture.

ADDS 100 may provide stimuli 180 to user 160 of user device 150 by presenting content matching conditions of task definitions 133. User 160 provides reactions 140 to stimuli 180 using devices attached to user device 150. For example, user 160 of user device 150 may react to a task presented to user 160 by clicking buttons or touching a screen to respond to stimuli 180. Reactions 140 to stimuli 180 associated with a task may include sharing the answer to the task. For example, a task may include a question that provides a list of reaction options that can be selected by user 160 in response to the question. In some embodiments, reactions 140 may include a non-reaction of a user for a task. Non-reactions may be recorded based on no response to stimuli after a certain time period. The time period to determine non-reactions to stimuli may be configurable by the user (e.g., user 160) of ADDS 100.

Task definitions 133 include rules of reaction that may be presented beforehand for user 160 of user device 150 to react to stimuli 180 based on rules of a task in task definitions 133. Rules may include when to react and how to react. In some embodiments, rules may include multiple conditional statements dividing and sub-dividing reaction types and reaction times based on content satisfying a task definition. In some embodiments, a task may include rules that extend the rules of another task.

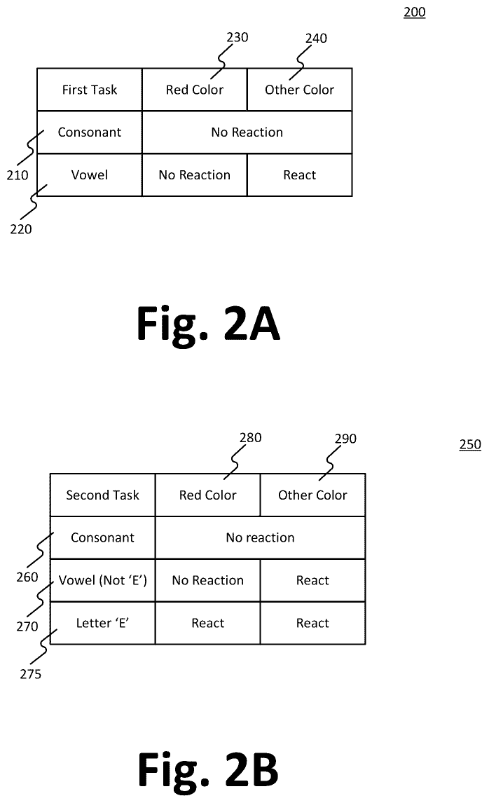

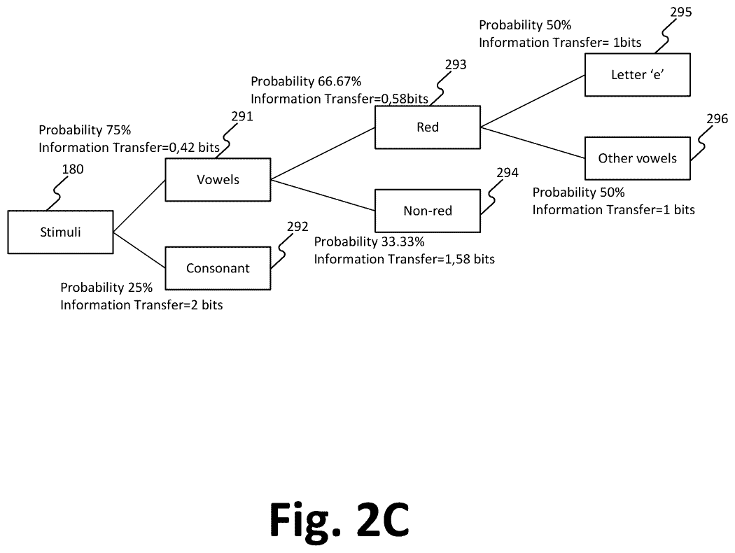

FIGS. 2A-B are tabular representations of exemplary tasks for detection of ADHD and its probabilities, according to some embodiments of the present disclosure. The tabular representations include various response reaction conditions presented as row and column headers 210-240 and 260-290. As illustrated in FIG. 2A, row headers 210 and 220 divide the possible content presented to a user (e.g., user 160 of FIG. 1) on a user device (e.g., user device 150 of FIG. 1). The divided content indicates when a user is supposed to react to the content presented as stimuli 180 on user device 150. For example, task 200 divides content into consonants and vowels with a rule to not react when consonants are presented as stimuli. A task may include additional conditions to apply restrictions to reactions to stimuli (e.g., stimuli 180 of FIG. 1) presented to a user. In task 200, additional conditions are included as column headers (e.g., column headers 230 and 240). The additional conditions may apply restrictions when the first condition for a reaction is met. For example, as illustrated in FIG. 2A, task 200 includes a second condition of letter color, which limits the reaction for vowels when it is a red-colored vowel.

Tasks may include multiple sub-conditions that add different restrictions or allowances. For example, FIG. 2B presents task 250 in a tabular form where vowels may include the condition to determine whether it is letter “E” or other vowels as one condition and whether the color of the letters is “Red” or an “Other Color” as a second condition.

Reactions are a user's (e.g., user 160) actions on a user device (e.g., user device 150). A reaction may include a positive reaction (e.g., clicking a button) and a negative reaction (e.g., clicking a different button). In some embodiments, a negative reaction may be no reaction. For example, in task 200, when a consonant is displayed (rule 210), user 160 is expected to have a negative reaction by not reacting to the displayed letter. A task condition may divide the possible universe of content into active and silent states. Active and silent states of a condition indicate when user 160 is expected to react or not react to an instance from the universe of content presented as stimuli 180. For example, as illustrated in FIG. 2A, task 200 includes the universe of letters as content and is divided into consonants associated with a negative reaction and vowels associated with a positive reaction. In some embodiments, a task may include restrictions on a condition to further restrict the expectation of an active state. For example, in task 200, condition 220 may have a limitation to not react when the displayed vowel is displayed in a red color. In some embodiments, the second condition may apply restrictions on content belonging to both active and silent states.

FIG. 2B illustrates a tabular representation of task 250 with additional conditions for reversal of restrictions on active state content. As illustrated in FIG. 2B, rules 260 and 270 include the active and silent states of the first condition, similar to rules 210 and 220 of the first condition in task 200. Similar to task 200, task 250 includes a second condition with rules 280 and 290 to place a restriction when the first condition matches the active state. A third condition can split the active state content besides the second condition. The third condition splits the active state content to reverse the restriction placed by second condition rules 280 and 290. For example, as illustrated in FIG. 2B, task 250 includes a third condition that splits the vowel content in the active state to the letter “E” and other vowels. When the third condition is positive, the task allows for reversal of the restriction on reaction placed on the active state content by the second condition. For example, in task 250, second condition rule 280 places a restriction to not react when the vowel is a “Red Color,” but rule 275 of the third condition reverses this restriction for the letter “E” by allowing reaction.

A user (e.g., user 160) of ADDS 100 may be presented with content belonging to the universe of content that meets the first, second, or third conditions as defined in tasks 200 and 250 for reaction along with rules of expected reactions. ADDS 100 may then collect the reactions (e.g., reactions 140 of FIG. 1) and signals generated in the brain of user 160 when submitting reactions 140. Brain signals collected on receiving user 160's reactions 140 on display of stimuli 180 are evaluated to determine the level of connectivity between regions of user 160's brain from where brain signals are collected. A detailed description of different brain regions and determination of levels of connectivity is provided in the description of FIG. 3 below.

FIG. 2C illustrates conditional probabilities of each output based on a cascade model, according to some embodiments of the present disclosure. The conditional probabilities and information transfer are calculated using the Transfer Information Content (TIC) metrics presented below. For example, using the TIC metric below, in task 200, when the consonant 292 data is observed by a user (e.g., user 160 of FIG. 1), the TIC metric calculates the information transfer amount using the TIC metric defined below as follows:

$$I ( a \vert s = consonant ) = I ( a ) - I ( s = consonant ) + I ( s = consonant \vert a ) = - {log}_{2} \frac{1}{4} - ( - {log}_{2} \frac{1}{4} ) - {log}_{2} 1 {0}^{- 5} = 2 - 2 + 16.61 = 16.61 bits$$

In the above scenario, I(s) «I(s|a) ⇒TIC(s→a)<0, i.e., there is a negative transfer of information from stimuli 180, including a display of a consonant as part of task 200 that causes a reaction. The negative information bits are an indication of inhibition or user 160 limiting himself/herself from providing a positive reaction (e.g., clicking a button). As a result, because I(a|s) is much larger than I(a), the outcome is not to press the button.

If vowel data (e.g., vowels 291) is presented as part of task 200 to user 160, then the user 160 needs to check whether the color of the letter is red (e.g., red vowel 293) or not. In the case of non-red vowel 294, the information transfer is calculated using the conditional TIC metric as follows:

$$I ( a \vert s , c ) = I ( a ) - I ( s = vowel ) + I ( s = vowel \vert a ) - I ( c = n o {n}_{r e d} \vert s = vowel ) + I ( c = n o {n}_{r e d} \vert a , s = vowel ) = - {log}_{2} \frac{1}{4} - ( - {log}_{2} \frac{3}{4} ) - {log}_{2} 1 - ( - {log}_{2} \frac{1}{3} ) - {log}_{2} 1 = 2 - 0.42 + 0 - 1.58 + 0 = 0 bits$$

Both sensorimotor and context TIC are positive and subtracted from the initial uncertainty, which is reduced to zero. Consequently, the button is pressed.

If red vowel data is presented as part of task 200 to user 160, then user 160 needs to check whether the letter is red. Applying conditional TIC to the context of a red color, we have:

$$I ( a \vert s , c ) = I ( a ) - I ( s = vowel ) + I ( s = vowel \vert a ) - I ( c = red \vert s = vowel ) + I ( c = red \vert a , s = vowel ) = - {log}_{2} \frac{1}{4} - ( - {log}_{2} \frac{3}{4} ) - {log}_{2} 1 - ( - {log}_{2} \frac{1}{3} ) - {log}_{2} {10}^{- 5} = 2 - 0.42 + 0 - 0.58 + 16.61 = 17.61 bits$$

In this case, the component I(c|a, s) generates negative bits of information that counteract the ones generated by the evidence representing the top-down control exerted by the subject. To derive the value of the probability P(c|a, s), whose information content is of interest, one can reason like this: suppose that a vowel has appeared and that the user (e.g., user 160 of FIG. 1) has pressed the button, then what is the probability of that vowel being red? The answer is that this probability is very low and, therefore, its associated information content is very high, i.e., a huge amount of negative bits of information have been generated in order to counteract the positive bits generated by the evidence I(c|s).

Therefore, in this contextual case, there is a negative transfer of information from the evidence of an appearance of a red vowel to the action a of pressing the button.

If red vowel data (e.g., red vowel data 293) is presented as part of task 250 to user 160, then user 160 needs to check whether the letter is “E” or not. The information transfer is calculated using an episodic TIC metric as follows:

$$I ( a \vert s , c , e ) = I ( a ) - I ( s = vowel ) + I ( s = vowel \vert a ) - I ( c = red \vert s = vowel ) + I ( c = red \vert a , s = vowel ) - I ( e \vert s = vowel , c = red ) + I ( e \vert a , s = vowel , c = red )$$

$$= - {log}_{2} \frac{1}{2} - ( - {log}_{2} \frac{3}{4} ) - {log}_{2} 1 - ( - {log}_{2} \frac{2}{3} ) - {log}_{2} \frac{1}{2} - ( - {log}_{2} \frac{1}{2} ) - {log}_{2} 1$$

$$= 1 - 0.42 + 0 - 0.58 + 1 - 1 + 0 = 0$$

The positive sensorimotor TIC reduces the uncertainty about the action, but the negative context TIC increases the uncertainty, and the net contribution is zero. Finally, the positive episode TIC further reduces the uncertainty and it is reduced to zero. Consequently, the button is pressed.

The amount of information bits described above can be used as a default value for comparing different users to determine attention levels. ADDS 100 may begin with these default values as expected signal values and revise them as signals from more individuals considered to be part of the control group are added.

FIG. 3 illustrates connection networks between regions of the brain, according to some embodiments of the present disclosure. ADDS 100 may identify the presence of signals in various brain regions representing different connection networks when a user (e.g., user 160 of FIG. 1) provides reactions 140. ADDS 100 may use identified signals in connection networks to determine the level of connectivity between different regions of the brain. ADDS 100 may evaluate the levels of connectivity between various regions of the brain to determine attention levels and whether the user is indicative of having ADHD based on those determined attention levels.

ADDS 100 may determine and utilize signals of varying frequencies to determine attention levels and deficiencies. ADDS 100 may group signals by frequency spectrum when evaluating signals and connectivity using signals to determine attention levels and attention deficiencies. ADDS 100 groups signals in the 8-12 Hz frequency range as an alpha group, signals in the 20-30 Hz frequency range as a beta group, and signals in the 4-8 Hz frequency range as a theta group. In some embodiments, ADDS 100 may collect signals of different frequencies at different times and from different regions of the brain.

Each of the signal groups indicates a certain aspect of task stimuli provision and reaction behavior. For example, the alpha band oscillation amplitude may indicate inhibition of activity. In some embodiments, signal group characteristics (e.g., amplitude) may have multiple meanings. For example, the same alpha band oscillation amplitude may also indicate active processing resulting in it being considered task-relevant information. Any change in signals or characteristics such as the amplitude of signals may be considered as a difference in the cognitive activity in the brain of user 160 generating signals.

In some embodiments, a decrease in connectivity between regions of the brain indicates an increase in attention. The increase of connectivity is based on tasks used to activate different regions of user 160's brain.

As illustrated in FIG. 3, the disclosed embodiments of ADDS 100 evaluate communication between various known regions of the brain (e.g., regions within the dorsal attention network (DAN) 310 or ventral attention network (VAN) 320) used for evaluating attention. The disclosed embodiments of ADDS 100 may utilize tasks defined in FIGS. 2A-B to generate information flow of positive and negative bits that takes place between the DAN 310 and VAN 320, and identify the pathway involved in the flow of this information. ADDS 100 may also identify alternate pathways and connectivity networks that may include signals when reacting to the same stimuli by different individuals. For example, ADDS 100 may detect signals in dorsoventral attention network (DVAN) 330.

ADDS 100 determines if a user (user 160 of FIG. 1) is indicative of having ADHD by determining and comparing the level of connectivity between regions of the brain generated in a user's brain to that of a known healthy group of individuals. The disclosed embodiments of ADDS 100 provide stimuli using tasks as ways to trigger activity within brain networks and generate signals by transfer of information (positive and negative bits), and then use the generated signal connectivity to test for ADHD in a user (e.g., user 160 of FIG. 1). For example, ADDS 100 may use tasks 200 and 250 to activate and test for the presence of signals in DVAN 330 by presenting instances of the universe of content defined by tasks 200 and 250 presented for user reaction. The usage of DVAN 330 is tested by comparing measured signals of different types as evaluated in user 160 to a previously identified healthy set of users (e.g., control group 131) with no ADHD. A detailed description of the comparative analysis of signal groups between different users is presented in the description of FIG. 5 below.

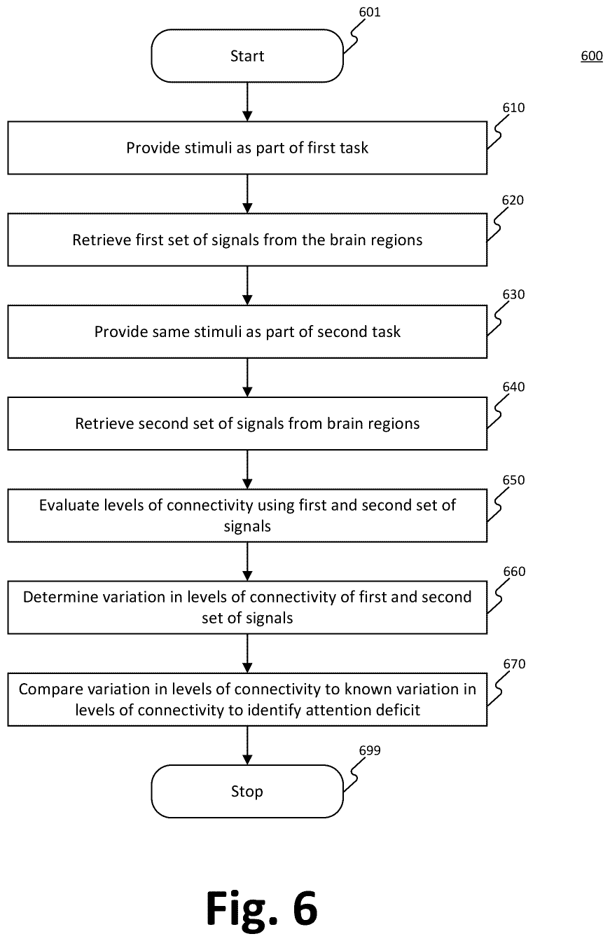

In some embodiments, ADHD may also be determined by comparing the variation in connectivity with switched tasks having different rules. In such a scenario, an instance of a universe of content is chosen that has opposite reactions defined in the tasks. For example, a red letter “E” presented as part of task 200 would result in a no-go condition with an active state restricted by a negative second condition. The same red letter “E” presented as part of task 250 results in a restricted second condition reversed by the third condition. A detailed description of the attention network analysis in task switching scenarios is presented in the description of FIG. 6 below.

Disclosed embodiments of ADDS 100 may help model the probability of ADHD levels based on the presented tasks for actions. The probability of ADHD levels helps accurately predict the presence of ADHD and the probability of ADHD. The model that identifies the probability of the level of ADHD can be implemented using example tasks 200 and 250 presented in the description of FIG. 2C above.

Bayes' Theorem establishes that the entire cortex represents probability distributions that only collapse onto estimates when decisions are needed. The effect of data x as stimuli 180 on user 160 under Bayes' Theorem to change the prior distribution P(θ) into the posterior distribution P(θ| x) is presented as follows:

$$P ( θ \vert x ) = \frac{P ( x \vert θ ) P ( θ )}{P ( x )} ,$$

where P(x) is known as the evidence and P(x|θ) is the likelihood of the Bayes' Theorem formula.

The same formula presented using −log functions used for representing information content transferred in the brain when data x is presented as stimuli 180 is as follows:

$$- {log}_{2} [ P ( θ \vert x ) ] = - {log}_{2} [ \frac{P ( x \vert θ ) P ( θ )}{P ( x )} ]$$

$$- {log}_{2} [ P ( θ \vert x ) ] = - {log}_{2} P ( x \vert θ ) - {log}_{2} P ( θ ) + {log}_{2} P ( x )$$

The information content (measured in bits) of the event x_i, also known as self-information, is given by:

I(xᵢ)=−log₂[p(xᵢ)]bits

So the above logarithmic equation represented as information bits is as follows:

I(θ|x)(bits)=I(θ)(bits)−I(x)(bits)+I(x|θ)(bits)

The key aspect of the above equation is that the mentioned change of information content of a single model θ in the space of models that provokes a single observation x that occurs on presenting data x is not compounded of single information content, but is instead two information contents I(x) and I(x|θ) that are generated by a single observation. Therefore, in the equation above, −I(x)(bits)+I(x|θ)(bits) represents the information transferred to a single model θ due to the observation x. ADDS 100 may calculate this information transfer using a novel metric called the Transfer Information Content (TIC), which may be defined as:

TIC(x→θ)(bits)=I(x)−I(x|θ)

I(θ) may also be interpreted as the a priori uncertainty about model θ, and I(θ|x) as the a posteriori uncertainty about the model θ after a single observation x.

When the TIC metric is applied to the information content equation above, it is transformed as follows:

I(θ|x)(bits)=I(θ)(bits)−TIC(x→θ)(bits)

ADDS 100 uses the TIC metric to describe the information flow. If the negative bits of the information content of the Bayes' Likelihood, I(θ|x), do not cancel the positive bits of the information content of the evidence, I(x), then the TIC metric is positive, i.e., there is a positive net transfer of information. On the contrary, if the negative bits corresponding to I(θ|x) are bigger than the bits of the evidence I(x), then the TIC is negative, and there is a negative net information transfer. A detailed description of the usage of this gain or loss of bits in form signal transfer between two regions to detect ADHD is provided in detail in the description of FIG. 5 below.

Using the above-defined TIC metric, the information content when represented using stimuli (e.g., stimuli 180 of FIG. 1) and action (e.g., reactions 140 of FIG. 1) transforms to

I(a|s)=I(a)−TIC(s→a)(bits)

I(a) may also be interpreted as the a priori uncertainty about the action a, and I(a|s) as the a posteriori uncertainty about the action a taken by a user (e.g., user 160 of FIG. 1) after stimulus s occurs.

The information content transfer that may trigger action a when the stimulus s occurs in a context c, which is defined in tasks 200 and 250 using different conditions, is as follows:

$$I ( a \vert s , c ) = - {log}_{2} [ P ( a \vert s , c ) ]$$

$$I ( a \vert s , c ) = - {log}_{2} [ P ( a \vert c , s ) ] = - {log}_{2} [ \frac{P ( a \vert s ) P ( c \vert a , s )}{P ( c \vert s )} ]$$

$$I ( a \vert s , c ) = - {log}_{2} [ P ( a \vert s ) ] - {log}_{2} [ \frac{P ( c \vert a , s )}{P ( c \vert s )} ]$$

ADDS 100 may use a conditional TIC metric to describe the contextual information content transfer. The information content is defined as follows:

I(a|s,c)=I(a)−TIC(s→a)−TIC(c→a|s)

I(a|s,c)=I(a)−TIC(s→a)(bits)−[I(c|s)−I(c|a,s)]=I(a)−I(s)+I(s|a)−I(c|s)+I(c|a,s)(bits)

I(a) may also be interpreted as the a priori uncertainty about the action a, and I(a|s, c) as the a posteriori uncertainty about the action a after stimulus s occurs in a context c.

ADDS 100 may use conditional TIC to describe transferred information bits when an additional condition, such as a second condition in task 200 defined using rules 230 and 240, is presented to user 160.

In some embodiments, ADDS 100 may consider the occurrence of stimulus s in a context c and in an episode (temporal context) e to compute the TIC metric for the information transfer that may trigger an action a. The information transfer is as follows:

I(a|s,c,e)=−log₂[P(a|s,c,e)]

Similar to the contextual case, ADDS 100 may then apply the conditioned version of Bayes' Theorem to the a posteriori probability as follows:

$$I ( a \vert s , c , e ) = - {log}_{2} [ P ( a \vert c , s , e ) ] = - {log}_{2} [ \frac{P ( a \vert s , c ) P ( e \vert a , s , c )}{P ( e \vert s , c )} ]$$

And then, applying logarithm rules, we have:

$$I ( a \vert s , c , e ) = - {log}_{2} [ P ( a \vert s , c ) ] - {log}_{2} [ \frac{P ( e \vert a , s , c )}{P ( e \vert s , c )} ]$$

ADDS 100 may use a conditional TIC metric to describe the episodic information content transfer. The episodic metric is defined as follows:

I(a|s,c,e)=I(a)−TIC(s→a)−TIC(c→a|s)−TIC(e→a|s,c)

Now, the sensorimotor TIC, the contextual conditional TIC, and the episodic conditional TIC can be broken down into their two components:

$$I ( a \vert s , c , e ) = I ( a ) - TIC ( s \to a ) - TIC ( c \to a \vert s ) - [ I ( e \vert s , c ) - I ( e \vert a , s , c ) ] = I ( a ) - I ( s ) + I ( s \vert a ) - I ( c \vert s ) + I ( c \vert a , s ) - i ( e \vert s , c ) + 1 ( e \vert a , s , c )$$

The components of the episodic conditional TIC, I(e|s, c) and I(e|a, s, c), represent bottom-up and top-down contributions in action selection, respectively. Following the reasoning of the contextual case, it may be possible that an uninformative stimulus in the given context may become informative if its episodic conditional TIC is positive and compensates the negativity of the contextual conditional TIC. ADDS 100 can also interpret I(a) as the a priori uncertainty about the action a, and I(a|s, c, e) as the a posteriori uncertainty about the action a after stimulus s occurs in a context c and in an episode e.

ADDS 100 may use a conditional TIC to describe transferred information bits when an additional condition, such as a second condition in task 250 defined using rules 280 and 290, is presented to user 160. A detailed description of conditional probabilities and bits of information transferred on presenting various stimuli to a user (e.g., user 160 of FIG. 1) is presented in the description of FIG. 2C above.

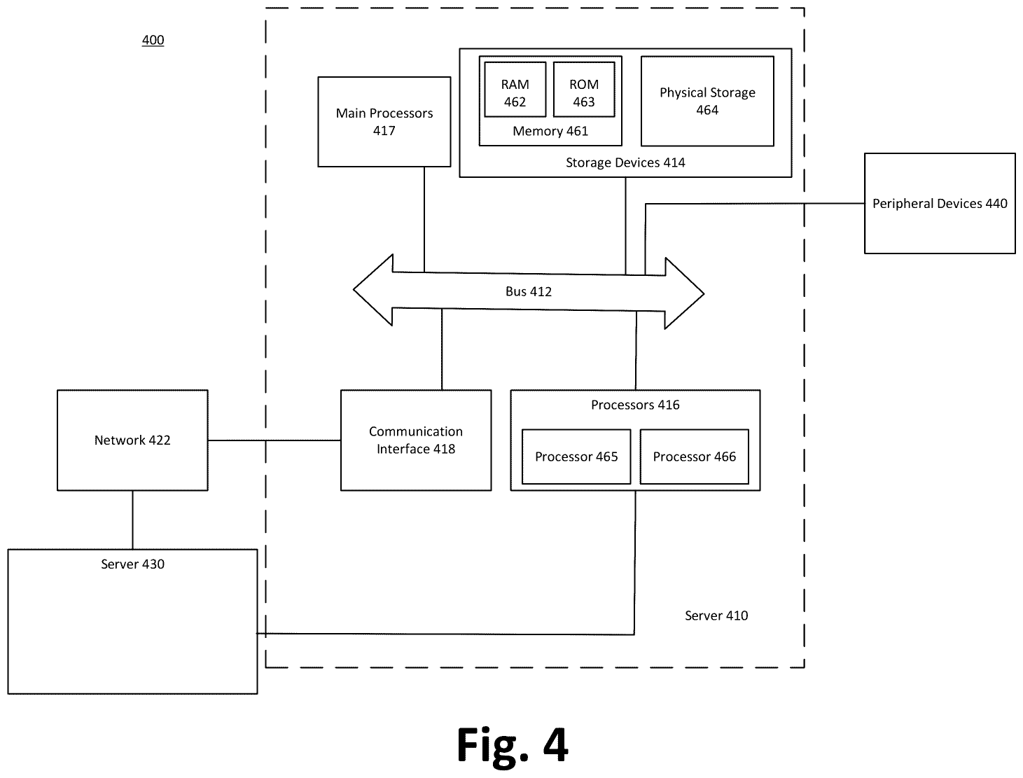

FIG. 4 illustrates a schematic diagram of an exemplary server of a distributed system, according to some embodiments of the present disclosure. According to FIG. 4, server 410 of distributed computing system 400 comprises a bus 412 or other communication mechanisms for communicating information, one or more processors 416 communicatively coupled with bus 412 for processing information, and one or more main processors 417 communicatively coupled with bus 412 for processing information. Processors 416 can be, for example, one or more microprocessors. In some embodiments, one or more processors 416 comprise processor 465 and processor 466, and processor 465 and processor 466 are connected via an inter-chip interconnect of an interconnect topology. Main processors 417 can be, for example, central processing units (CPUs).

Server 410 can transmit data to or communicate with another server 430 through a network 422. Network 422 can be a local network, an internet service provider, Internet, or any combination thereof. Communication interface 418 of server 410 is connected to network 422, which can enable communication with server 430. In addition, server 410 can be coupled via bus 412 to peripheral devices 440, which comprise displays (e.g., cathode ray tube (CRT), liquid crystal display (LCD), touch screen, etc.) and input devices (e.g., keyboard, mouse, soft keypad, etc.).

Server 410 can be implemented using customized hardwired logic, one or more application-specific integrated circuits (ASICs) or field-programmable gate arrays (FPGAs), firmware, or program logic that, in combination with the server, causes server 410 to be a special-purpose machine.

Server 410 further comprises storage devices 414, which may include memory 461 and physical storage 464 (e.g., hard drive, solid-state drive, etc.). Memory 461 may include random access memory (RAM) 462 and read-only memory (ROM) 463. Storage devices 414 can be communicatively coupled with processors 416 and main processors 417 via bus 412. Storage devices 414 may include a main memory, which can be used for storing temporary variables or other intermediate information during the execution of instructions to be executed by processors 416 and main processors 417. Such instructions, after being stored in non-transitory storage media accessible to processors 416 and main processors 417, render server 410 into a special-purpose machine that is customized to perform operations specified in the instructions. The term “non-transitory storage media” as used herein refers to any non-transitory media storing data or instructions that cause a machine to operate in a specific fashion. Such non-transitory storage media can comprise non-volatile media or volatile media. Non-transitory storage media include, for example, optical or magnetic disks, dynamic memory, floppy disks, flexible disks, hard disks, solid-state drives, magnetic tapes, or any other magnetic data storage medium; CD-ROMs or any other optical data storage medium; any physical medium with patterns of holes; RAMs, PROMs, EPROMs, FLASH-EPROMs, NVRAMs, flash memory, registers, caches, or any other memory chip or cartridge; and networked versions of the same.

Various forms of media can be involved in carrying one or more sequences of one or more instructions to processors 416 or main processors 417 for execution. For example, the instructions can initially be carried out on a magnetic disk or solid-state drive of a remote computer. The remote computer can load the instructions into its dynamic memory and send the instructions over a telephone line using a modem. A modem local to server 410 can receive the data on the telephone line and use an infra-red transmitter to convert the data to an infra-red signal. An infra-red detector can receive the data carried in the infra-red signal, and appropriate circuitry can place the data on bus 412. Bus 412 carries the data to the main memory within storage devices 414, from which processors 416 or main processors 417 retrieve and execute the instructions.

ADDS 100 or one or more of its components may reside on either server 410 or 430 and may be executed by processors 416 or main processors 417. In some embodiments, the components of ADDS 100 may be spread across multiple servers 410 and 430. For example, measurement module 110 and report module 120 (of FIG. 1) may be executed on multiple servers. Similarly, database 130 may be maintained by multiple servers 410 and 430.

FIG. 5 is a flowchart depicting operations of an exemplary method for detection of ADHD, according to some embodiments of the present disclosure. The steps of method 500 may be performed by ADDS 100 for purposes of illustration. It will be appreciated that the illustrated method 500 may be altered to modify the order of steps and to include additional steps.

In step 510, ADDS 100 may provide stimuli to a user (e.g., user 160 of FIG. 1) that causes a reaction. ADDS 100 may provide stimuli by presenting a task (e.g., tasks 200 and 250 of FIG. 2) on a user device (e.g., user device 150 of FIG. 1). ADDS 100 may present a task by displaying the rules of a task followed by content to review and cause a reaction. User 160 of user device 150 may react to provided stimuli which is an instance of possible content based on rules of a task. ADDS 100 may continuously provide stimuli by presenting content matching a task. In some embodiments, ADDS 100 may loop through different tasks to provide stimuli to the user 160 of ADDS 100.

In step 520, ADDS 100 may retrieve signals from sensors for presented stimuli. Sensors can be probes from an electroencephalography (EEG) or magnetoencephalography (MEG) device, or other functional neuroimaging technique for mapping brain activity like functional Magnetic Resonance Imaging (fMRI) and functional Near-Infrared Spectroscopy (fNIRS). Sensors are placed on the head matching different known regions (e.g., brain regions 310-330 of FIG. 3) of the brain. ADDS 100 may retrieve signals based on certain trigger events. In some embodiments, trigger events may include a period of time. The period of time may include a wait time since a stimulus was provided in step 510. ADDS 100 may allow the configuration of trigger events by allowing the selection of trigger events to retrieve signals and parameters of the trigger events. For example, ADDS 100 may allow the amount of wait time after providing a stimulus before retrieving signals from different brain regions. The EEG/MEG device may be a standard EEG or MEG device. The EEG/MEG device may measure a plurality of signals of respective source positions located inside a cranial cavity of user 160. Each source position may correspond to a known brain area, such as a lobe, a lobule, a sulcus, or a gyrus. In some examples, the plurality of signals may be measured while the subject is performing one or more tasks that may be presented to user 160 by ADDS 100. In one or more embodiments, a signal may correspond to the measurement of an electrode of an EEG device or to the measurement of a sensor coil of an MEG device. In some embodiments, the signal may correspond to an aggregated signal (such as an average signal) over the measurements of multiple electrodes (when an EEG is used) or multiple sensor coils (when an MEG is used). Such an aggregated signal may correspond to an area of the brain that comprises the respective positions of the signals that are being aggregated in the aggregated signal.

MEG signals can be measured using a 306-channel (102 magnetometers and 204 planar gradiometers) whole-head MEG Elekta Neuro image system. In some embodiments, brain signals can be measured using an online anti-alias bandpass filter between 0.1 and 330 Hz and a sampling rate of 1,000 Hz. In some embodiments, a user's (e.g., user 160 of FIG. 1) head shape may be acquired using a three-dimensional Fastrak digitizer (Polhemus, Colchester, Vermont), in addition to three fiducial points (nasion and left and right preauricular points) as landmarks. In another embodiment, four Head Position Indicator (HPI) coils may be recorded on the user 160's scalp (forehead and the mastoids). In some embodiments, two sets of bipolar electrodes may be used to record eye blinks and heart beats, respectively.

In step 530, ADDS 100 may evaluate signals of each brain region from the retrieved brain signals of step 520. ADDS 100 may evaluate signal quality to remove artifacts from signals and reconstruct the brain activity using signals retrieved from each sensor on the user's head approximately representing each brain region. ADDS 100 may improve data by removing external noise, such as ocular, cardiac, or muscle artifacts, from the EEG/MEG data measured, and segmenting the EEG/MEG data such that the time series may have the same length (e.g., 1 second).

In step 531, ADDS 100 may filter the extracted signals in step 530 into multiple frequency bands by using, for example, bandpass filters. The frequency bands may be functionality-relevant signal bands. As provided in the description of FIG. 3 above, a signal may be filtered into different frequencies to indicate different attention behaviors of users of ADDS 100. For example, ADDS 100 extracts signals into frequency bands: theta (4 to 8 Hz), alpha (8 to 12 Hz), low beta (12 to 20 Hz), high beta (20 to 30 Hz), and low gamma (30 to 45 Hz). In some embodiments, the frequency ranges describing theta, alpha, low beta, high beta, and low gamma bands may be different. In some embodiments, there may be additional groups of signals in between the example frequency bands. ADDS 100 may allow configuration of frequency band ranges to be part of a signal group. ADDS 100 extracts signals by frequency bands by filtering and removing signals not falling into one of the selected frequency bands.

In step 532, ADDS 100 may determine the level of connectivity between two brain regions based on dependency measures between signals of two brain regions for each frequency band. For example, ADDS 100 may measure the phase synchronization between signals grouped under a frequency band to calculate the connectivity value. Phase synchronization measures include Phase-Lag Index or Phase-Locking Value (PLV) and its derivatives, imaginary part of PLV (iPLV) and corrected imaginary part of PLV (ciPLV). ADDS 100 may use other different connectivity measures, including Information Theory measures like Mutual Information or Transfer Entropy, Granger causality measures, and classical measures like Correlation, Cross-Correlation, Coherence, or Phase-Slope Index. ADDS 100 may measure the level of connectivity between two brain regions based on metrics derived from TIC. In some embodiments, dependency measures may be based on connectivity between brain regions between different frequency bands.

In step 533, ADDS 100 may check if the type of signal matches theta band signal frequencies by filtering theta band signals. If the answer is “No,” then jump to step 538.

In step 534, ADDS 100 may check if the first condition of the second task is met. If the answer to the question is “Yes,” then jump to step 535. If the answer to the question is “No then jump to step 556.

In step 535, ADDS, 100 may check if second condition of second task is met. If the answer to the question is ‘No,” then jump to step 537.

In step 536, ADDS 100 may check if the third condition of the second task is met. If the answer to the question is “Yes,” then proceed to step 537. If the answer to the question is “No,” then jump to step 556.

In step 537, ADDS 100 may check whether the level of connectivity of the user as calculated in step 532 is less than the level of connectivity of a healthy control group. If the answer to the question is “Yes,” then jump to step 570. ADDS 100 may retrieve the level of connectivity of the healthy control group stored in population database 130. ADDS 100 may receive control group type user as input and stores the results in control group 131. ADDS 100 may compare the level of connectivity by checking whether the calculated connectivity level is less than the range of connectivity levels possible for the healthy control group. ADDS 100 may determine the range of values of the connectivity level of a healthy control group based on the level of connectivity of an individual identified to have ADHDas evaluated by method 500.

In step 538, ADDS 100 may check if the type of signal matches beta band signal frequencies by filtering beta band signals. If the answer is “No,” then jump to step 549.

In step 539, ADDS 100 may check if the stimulus is from the first task. If the answer to the question is “No,” then jump to step 544.

In step 540, ADDS 100 may check if the first condition of the first task is met. If the answer to the question is “No,” then jump to step 548.

In step 541, ADDS 100 may check if the second condition of the first task is met. If the answer to the question is “Yes,” then jump to step 548. If the answer to the question is “No,” then jump to step 556.

In step 544, ADDS 100 may check if the first condition of the second task is met. If the answer to the question is “Yes,” then proceed to step 545. If the answer to the question is “No,” then jump to step 556.

In step 545, ADDS 100 may check if the second condition of the second task is met. If the answer to the question is “Yes,” then proceed to step 546. If the answer to the question is “No,” then jump to step 547.

In step 546, ADDS 100 may check if the third condition of the second task is met. If the answer to the question is “Yes,” then jump to step 548. If the answer to the question is “No,” then jump to step 556.

In step 547, ADDS 100 may check if the third condition of the second task is met. If the answer to the question is “No,” then proceed to step 548. If the answer to the question is “Yes,” then jump to step 556.

In step 548, ADDS 100 may check whether the level of connectivity of the user as calculated in step 532 is greater than the level of connectivity of the healthy control group. If the answer to the question is “Yes,” then jump to step 570. ADDS 100 may retrieve the level of connectivity of the healthy control group stored in population database 130. ADDS 100 may receive control group type user as input and stores the results in control group 131. ADDS 100 may compare the level of connectivity by checking whether the calculated connectivity level is less than the range of connectivity levels possible for the healthy control group. ADDS 100 may determine the range of values of the connectivity level of a healthy control group based on the level of connectivity of an individual identified to have ADHD as evaluated by method 500.

In step 549, ADDS 100 may check if the type of signal matches alpha band signal frequencies by filtering alpha band signals. If the answer to the question is “Yes,” then proceed to step 550. If the answer to the question is “No,” then jump to step 556.

In step 550, ADDS 100 may check if the stimulus is from the first task. If the answer to the question is “No,” then jump to step 554.

In step 551, ADDS 100 may check if the first condition of first task is met. If the answer to the question is “No,” then proceed to step 551.2; otherwise, jump to step 552.

In step 551.2, if the level of connectivity of user 160 as calculated in step 532 is stored in a container A1, then jump to step 555. Container A1 may be a data structure, such as a list or an array. In some embodiments, container A1 may be persisted on a disk in a flat file or a database. In step 552, ADDS 100 may check if the second condition of the first task is met. If the answer to the question is “No,” then proceed to step 552.2; otherwise, jump to step 552.3.

In step 552.2, if the level of connectivity of the user as calculated in step 532 is stored in a container A2, then jump to step 555. Container A2 may be a data structure, such as a list or an array. In some embodiments, container A2 may be persisted on a disk in a flat file or a database. Container A2 will contain alpha band connectivity values between brain areas when the first condition of the first task is met and the second condition is not met.

In step 552.3, the level of connectivity of user 160 as calculated in step 532 is stored in a container A1.

In step 553, ADDS 100 may check if the third condition of the first task is met, i.e., it is a red-colored vowel “E.” If the answer to the question is “Yes,” then proceed to step 553.2; otherwise, jump to step 555.

In step 553.2, if the level of connectivity of the user as calculated in step 532 is stored in a container A3, then jump to step 555. Container A3 may be a data structure, such as a list or an array. In some embodiments, container A3 may be persisted on a disk in a flat file or a database. Container A3 will contain alpha band connectivity values between brain areas when the first, second, and third conditions of the first task are met, i.e., it is a red-colored vowel “E.”

In step 554, ADDS 100 may check if the first, second, and third conditions of the second task are met. If the answer to the question is “Yes,” then proceed to step 554.2; otherwise, jump to step 556.

In step 554.2, if the level of connectivity of the user as calculated in step 532 is stored in container A4, then proceed to step 555. Container A4 may be a data structure, such as a list or an array. In some embodiments, container A4 may be persisted on a disk in a flat file or a database. Container A4 will contain alpha band connectivity values between brain areas when the first, second, and third conditions of the second task are met, i.e., it is a red-colored vowel “E.”

In step 555, ADDS 100 may check whether the level of connectivity of the user as calculated in step 532 is less than the level of connectivity of the healthy control group. If the answer to the question is “Yes,” then jump to step 570. ADDS 100 may retrieve the level of connectivity of the healthy control group stored in population database 130. ADDS 100 may receive control group type user as input and stores the results in control group 131. ADDS 100 may compare the level of connectivity by checking whether the calculated connectivity level is less than the range of connectivity levels possible for the healthy control group. The range of values to include for the connectivity level of a healthy control group may be determined based on the level of connectivity of an individual identified to have ADHD.

In step 556, ADDS 100 may check if all connectivity values calculated in step 532 have been checked for ADHD identification by going through steps 533 to 555. If the answer to the question is “Yes,” then proceed to step 557. If the answer to the question is “No,” the next connectivity value is checked by jumping to step 533.

In step 557, ADDS 100 may retrieve connectivity level values in containers A1 and A2 from steps 551.2, 552.2, and 552.3. ADDS 100 may compare the change in connectivity levels. If the reduction in the connectivity level is less than the reduction in the connectivity level of the healthy control group, then jump to step 570.

In step 558, ADDS 100 may retrieve connectivity level values in containers A3 and A4 from steps 553.2 and 554.2, respectively. ADDS 100 may compare the change in connectivity levels. If the variation in the connectivity level between brain regions of a user is different than the reduction in the connectivity level between the same regions of a healthy control group, then jump to step 570.

In some embodiments, ADDS 100 may compare the variation in the level of connectivity between two regions based on levels of connectivity based on signals determined in steps 533, 538, and 549 of method 500. ADDS 100 may calculate the probability of ADHD based on the difference in the variation of level connectivity between the user of ADDS 100 and the healthy control group. ADDS 100 may collect multiple levels of connectivity of the same user or a set of users to save them as healthy control group 131 data or for those identified with ADHD as users 132. ADDS 100 may use the stored values of levels of connectivity to determine the variation in the level of connectivity. In some embodiments, the variations in the levels of connectivity may only include observed levels of connectivity for the same data for a different condition. For example, a user of ADDS 100 may be presented with the same data and then presented with the rules of the task to determine levels of connectivity for the same data across multiple tasks. In step 560, ADDS 100 may mark user (e.g., user 160) whose brain signals are evaluated for level of connectivity to have an indication of ADHD. ADDS 100, upon completion of step 560, completes (step 599) executing method 500.

FIG. 6 is a flowchart depicting operations of an exemplary method for detection of ADHD, according to some embodiments of the present disclosure. The steps of method 600 may be performed by ADDS 100 for purposes of illustration. It will be appreciated that the illustrated method 600 may be altered to modify the order of steps and to include additional steps.

In step 610, ADDS 100 may provide stimuli to a user (e.g., user 160 of FIG. 1) that causes a reaction that match a first task (e.g., task 200 of FIG. 2A). ADDS 100 may select a set of tasks for testing a user (e.g., user 160 of FIG. 1) for ADHD that can satisfy rules (e.g., rules 220 and 230 of task 200, rules 275 and 280 of task 250). ADDS 100 may provide stimuli by displaying content that satisfies the rules of task 200 on a user device (e.g., user device 150 of FIG. 1).

In step 620, ADDS 100 may retrieve a first set of signals from brain regions. ADDS 100 may retrieve signals from different brain regions by collecting electric charges from probes attached to the head of user 160 being tested for ADHD. ADDS 100 may retrieve signals when a user reacts to the stimuli provided in step 610 by performing an action. Action performance may include clicking a button or selecting something presented on the screen using a pointing device.

In step 630, ADDS 100 may provide the same stimuli as part of a second task (e.g., task 250 of FIG. 2B). ADDS 100 may select tasks where the rules are satisfied by the first and second tasks. ADDS 100 may only select tasks from task definitions 133 where the same content satisfies different rules requiring opposite user reaction. For example, when a red-colored letter “E” is presented as a stimulus under task 200, then rules 220 and 230 are satisfied, and the user is expected not to react, but under task 250, rules 275 and 280 are satisfied, and the user is expected to react.

In step 640, ADDS 100 may retrieve a second set of signals from brain regions. Similar to step 620, ADDS 100 may retrieve signals by collecting brain images or electric charges from user 160's brain upon viewing stimuli in step 630. The signal may be generated as part of the reaction performed by user 160.

In step 650, ADDS 100 may evaluate levels of connectivity using the first and second set of signals. ADDS 100 may evaluate levels of connectivity by measuring the signals in certain regions of the brain (e.g., dorsal attention network (DAN) 310 of FIG. 3) and measuring the signal in other regions of the brain (e.g., ventral attention network (VAN) 320 of FIG. 3) to determine the amount of transferred signals indicating the level of connectivity.

In step 660, ADDS 100 may determine variations in the levels of connectivity of the first and second set of signals. ADDS 100 may determine variations in the levels of connectivity by calculating the levels of connectivity by providing multiple stimuli as part of the first and second tasks in steps 610 and 630, and retrieving signals of user 160's reaction in steps 620 and 640.

In step 670, ADDS 100 may compare variations in the levels of connectivity to known variations in the levels of connectivity to identify ADHD. ADDS 100 may compare variations in the levels of connectivity calculated in step 660 to variations in the levels of connectivity of a control group (e.g., control group 131). ADDS 100 may retrieve variations in the levels of connectivity of control group 131 from previously generated performance metrics 134. In some embodiments, ADDS 100 may need to evaluate the variations in the levels of connectivity from previously calculated levels of connectivity stored in performance metrics 134. ADDS 100 may indicate that user 160 has ADHD if the variations of the levels of connectivity of user 160 are different than the variations of the levels of connectivity of control group 131. ADDS 100 may consider the variations of the levels of connectivity to be different if the range of values of the levels of connectivity is different. In some embodiments, the variations of levels of connectivity are considered different if the ranges of values do not overlap. ADDS 100 may store attention deficit results in database 130 under users 132. ADDS 100, upon completion of step 670, completes (step 699) executing method 600.

Various operations or functions are described herein, which may be implemented or defined as software code or instructions. Such content may be directly executable (“object” or “executable” form), source code, or difference code (“delta” or “patch” code). Software implementations of the embodiments described herein may be provided via an article of manufacture with the code or instructions stored thereon, or via a communication interface method to send data via the communication interface. A machine or computer-readable storage medium may cause a machine to perform the functions or operations described and includes any mechanism that stores information in a form accessible by a machine (e.g., computing device, electronic system, and the like), such as recordable/non-recordable media (e.g., read only memory (ROM), random access memory (RAM), magnetic disk storage media, optical storage media, flash memory devices, and the like). A communication interface includes any mechanism that interfaces with any of a hardwired, wireless, optical, or similar medium to communicate with another device, such as a memory bus interface, a processor bus interface, an Internet connection, a disk controller, and the like. The communication interface may be configured by providing configuration parameters and/or sending signals to prepare the communication interface to provide a data signal describing the software content. The communication interface may be accessed via one or more commands or signals sent to the communication interface.

The present disclosure also relates to a system for performing the operations herein. This system may be specially constructed for the required purposes, or it may comprise a general-purpose computer selectively activated or reconfigured by a computer program stored in the computer. Such a computer program may be stored in a computer-readable storage medium, such as, but not limited to, any type of disk, including floppy disks, optical disks, CDROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Embodiments of the present disclosure may be implemented with computer-executable instructions. The computer-executable instructions may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments may include different computer-executable instructions or components having more or less functionality than illustrated and described herein.

Computer programs based on the written description and methods of this specification are within a software developer's skill. The various programs or program modules may be created using a variety of programming techniques. For example, program sections or program modules may be designed by means of JavaScript, Scala, Python, Java, C, C++, assembly language, or any such programming languages, as well as data-encoding languages (such as XML, JSON, etc.), query languages (such as SQL), presentation-related languages (such as HTML, CSS, etc.), and data transformation languages (such as XSL). One or more of such software sections or modules may be integrated into a computer system, non-transitory computer-readable media, or existing communications software.

The words “comprising,” “having,” “containing,” and “including,” and other similar forms are intended to be equivalent in meaning and be interpreted as open ended, in that an item or items following any one of these words is not meant to be an exhaustive listing of such item or items, or meant to be limited to only the listed item or items. In addition, the singular forms “a,” “an,” and “the” are intended to include plural references, unless the context clearly dictates otherwise.

Having described aspects of the embodiments in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

What is claimed is:

1. A non-transitory computer-readable medium including instructions that, when executed by at least one processor, cause the at least one processor to perform operations for automated detection of attention deficit hyperactivity disorder (ADHD), the operations comprising:
providing one or more stimuli to a user to activate a plurality of brain regions representing an attention network, wherein the one or more stimuli include displaying information with a first condition that causes a reaction and a second condition as an exception to the first condition;
retrieving a plurality of signals from the plurality of brain regions for each of the one or more stimuli, wherein each signal of the plurality of signals is accessed over a period of time, wherein the period of time starts when the information is displayed and ends when the reactions of the user are captured; and
evaluating the plurality of signals to detect ADHD based on a level of connectivity within an attention network when each signal of the plurality of signals corresponds to the one or more stimuli.

2. The non-transitory computer-readable medium of claim 1, wherein displaying information with a first condition that causes a reaction and a second condition as an exception to the first condition further comprises:
presenting an instance of data matching a first task, wherein the first task includes the first condition for expectation of an active state and a silent state of a first reaction, the second condition restricting the expectation of the active state of the first reaction.

3. The non-transitory computer-readable medium of claim 2, wherein the first reaction further comprises:
displaying the information on a screen meeting the first condition for expectation of the active state and the silent state of the first reaction; and
waiting for a threshold period for any reaction shared by the user.

4. The non-transitory computer-readable medium of claim 2, wherein the first reaction is at least one of clicking a pointing device, pressing a button, or taking no action for a time threshold.

5. The non-transitory computer-readable medium of claim 2, wherein evaluating the plurality of signals to detect ADHD based on the level of connectivity within the attention network further comprises:
filtering alpha band oscillation signals of two or more regions of the plurality of brain regions of the user upon receiving the first reaction and determining connectivity between two regions of the brain using the alpha band oscillation signals; and
comparing the level of connectivity between the two regions of the brain of the user to the level of connectivity of a healthy control group of users when the first condition of the first task satisfies the active state, and the second condition is negative, wherein if the level of connectivity of the user is less than the level of connectivity of the healthy control group of users, then the user is indicative of having ADHD.

6. The non-transitory computer-readable medium of claim 2, wherein evaluating the plurality of signals to detect ADHD based on the level of connectivity within the attention network further comprises:
filtering alpha band oscillation signals of two or more regions of the plurality of brain regions of the user upon receiving the first reaction and determining connectivity between two regions of the brain using the alpha band oscillation signals; and
comparing the level of connectivity between the two regions of the brain of the user to the level of connectivity of a healthy control group of users when the first condition of the first task satisfies the silent state, the second condition is positive, wherein if the level of connectivity of the user is less than the level of connectivity of the healthy control group of users, then the user is indicative of having ADHD.

7. The non-transitory computer-readable medium of claim 2, the operations further comprising:
filtering beta band oscillation signals of two or more brain regions upon receiving the first reaction and determining connectivity between two regions of the brain using the beta band oscillation signals; and
comparing the level of connectivity between the two regions of the brain of the user to the level of connectivity of a healthy control group of users when the first condition of the first task satisfies the silent state, the second condition is positive, wherein if the level of connectivity of the user is greater than the level of connectivity of the beta band oscillation signals of the healthy control group of users, then the user is indicative of having ADHD.

8. The non-transitory computer-readable medium of claim 2, the operations further comprising:
filtering alpha band oscillation signals of two or more brain regions upon receiving the first reaction and determining connectivity between two regions of the brain using the alpha band oscillation signals;
determining the level of connectivity between the two regions of the brain of the user when the first condition of the first task satisfies the silent state and the first condition of the first task satisfies the active state; and
comparing a reduction in the level of connectivity of the user between when the first task satisfies the silent state and when the first task satisfies the active state to a reduction in the level of connectivity of a healthy control group of users between when the first task satisfies the silent state and when the first task satisfies the active state, wherein if the reduction in the level of connectivity of the user is less than the reduction in the level of connectivity of the healthy control group of users, then the user is indicative of having ADHD.

9. The non-transitory computer-readable medium of claim 1, wherein evaluating the plurality of signals to detect ADHD based on the level of connectivity within the attention network further comprises:
combining signal data of each of the plurality of signals, wherein combining signal data includes determining an increase in the level of connectivity compared to an existing level of connectivity between regions from the plurality of regions or a reduction in the level of connectivity from the existing level of connectivity of the region of the plurality of regions.

10. The non-transitory computer-readable medium of claim 1, wherein displaying information with a first condition includes displaying a category of text or graphic.

11. The non-transitory computer-readable medium of claim 10, wherein the second condition includes displaying the category of text or graphic in a particular color.

12. The non-transitory computer-readable medium of claim 1, wherein the one or more stimuli further comprise:
displaying second information with the first condition that causes a reaction, the second condition as the exception to the first condition, and a third condition as an exception to the second condition.

13. The non-transitory computer-readable medium of claim 12, wherein displaying second information with the first condition that causes a reaction, the second condition as the exception to the first condition, and a third condition as an exception to the second condition further comprises:
presenting an instance of data matching a second task, wherein the second task includes the first condition for expectation of an active state and a silent state of a second reaction, the second condition restricting expectation of the active state of the second reaction, and the third condition that is an exception to the second condition for expectation of silent state of the second reaction.

14. The non-transitory computer-readable medium of claim 13, wherein the second reaction further comprises:
displaying information on a screen meeting the first condition for expectation of the active state and the silent state of the second reaction; and
waiting for a threshold period for any input shared by the user.

15. The non-transitory computer-readable medium of claim 13, wherein the third condition includes:
selecting a sub-category of a category of text or graphic meeting the first condition; and
displaying the sub-category of the text or graphic.

16. The non-transitory computer-readable medium of claim 13, wherein the second reaction is at least one of clicking a pointing device, pressing a button, or taking no action for a threshold time.

17. The non-transitory computer-readable medium of claim 13, the operations further comprising:
filtering beta band oscillation signals of two or more regions of the plurality of brain regions of the user upon receiving the second reaction and determining connectivity between two regions of the brain using the beta band oscillation signals; and
comparing the level of connectivity between the two regions of the brain of the user to the level of connectivity of a healthy control group of users when the first condition of the second task satisfies the active state, the second condition is negative and the third condition is negative, or when the first condition of the second task satisfies the active state, the second condition is positive and the third condition is positive, wherein if the level of connectivity of the user is greater than the level of connectivity of the healthy control group of users, then the user is indicative of having ADHD.

18. The non-transitory computer-readable medium of claim 13, the operations further comprising:
filtering theta band oscillation signals of two or more regions of the plurality of brain regions of the user upon receiving the second reaction from the user and determining connectivity between two regions of the brain using the theta band oscillation signals; and
comparing the level of connectivity between the two regions of the brain of the user to the level of connectivity of a healthy control group of users when the first condition of the second task satisfies the active state, the second condition is negative and the third condition is negative, or when the first condition of the second task satisfies the active state, the second condition is positive and the third condition is positive, wherein if the signal data of the level of connectivity of the user is less than the level of connectivity of the healthy control group of users, then the user is indicative of having ADHD.

19. The non-transitory computer-readable medium of claim 13, the operations further comprising:
filtering alpha band oscillation signals of two or more regions of the plurality of brain regions of the user upon receiving the second reaction from the user and determining connectivity between two regions of the brain using the alpha band oscillation signals;
determining a variation in the level of connectivity between the two regions of the brain of the user when presenting the instance of data for the second task where the first condition of the second task satisfies the active state, the second condition of the second task is positive and the third condition of the second task is positive to the level of connectivity between the two regions of the brain of the user when presenting the instance of data for the first task where the first condition of the first task satisfies the active state, and the second condition of the first task is positive; and
comparing the variation in the level of connectivity of the user to the variation in the level of connectivity of a healthy control group of users, wherein if the variation in the level of connectivity of the user is different than the variation in the level of connectivity of the healthy control group of users, then the user is indicative of ADHD.

20. The non-transitory computer-readable medium of claim 13, the operations further comprising:
filtering alpha, beta, and theta band oscillation signals of two or more regions of the plurality of brain regions of the user upon receiving the first reaction and the second reaction and determining connectivity between regions of the brain using the alpha, beta, and theta band oscillation signals; and
evaluating the probability of the detection of ADHD based on the comparison of the level of connectivity, reduction in the level of connectivity, and the variation in the level of connectivity between the regions of the brain of the user to the level of connectivity of a healthy control group of users when varying the first condition, the second condition of the first task, and the first condition, the second condition, and the third condition of the second task.

21. A computer-implemented method for automated detection of attention deficit hyperactivity disorder (ADHD), the method comprising:
providing one or more stimuli to a user to activate a plurality of brain regions representing an attention network, wherein the one or more stimuli include displaying information with a first condition that causes a reaction and a second condition as an exception to the first condition;
retrieving a plurality of signals from the plurality of brain regions for each of the one or more stimuli, wherein each signal of the plurality of signals is accessed over a period of time, wherein the period of time starts when the information is displayed and ends when the reactions of the user are captured; and
evaluating the plurality of signals to detect ADHD based on a level of connectivity in the attention network when each signal of the plurality of signals corresponds to the one or more stimuli.

22. An attention deficit detection system, comprising:
one or more memory devices storing processor-executable instructions; and
one or more processors configured to execute instructions to cause the attention deficit detection system to perform operations comprising:
providing one or more stimuli to activate a plurality of brain regions representing an attention network, wherein the one or more stimuli include displaying information with a first condition that causes a reaction and a second condition as an exception to the first condition;
retrieving a plurality of signals from the plurality of brain regions for each of the one or more stimuli, wherein each signal of the plurality of signals is accessed over a period of time, wherein the period of time starts when the information is displayed and ends when the reactions of the user are captured; and
evaluating the plurality of signals to detect ADHD based on a level of connectivity in the attention network when a signal of the plurality of signals corresponds to the one or more stimuli.

23. An attention deficit detection system, comprising:
one or more memory devices storing processor-executable instructions; and
one or more processors configured to execute instructions to cause the attention deficit detection system to perform operations comprising:
receiving first signal data from a user;
determining a first plurality of time series based on the first signal data, wherein each of the first plurality of time series corresponds to a respective source position located inside a cranial cavity of the user;
calculating a first correlation value for a first pair of time series, the first pair of time series being included in the determined first plurality of time series;
generating a score based on the first correlation value, the score being indicative of a patient having a cognitive impairment, such as an attention deficiency or ADHD; and
outputting the generated score.
